(19) United States
(12) Patent Application Publication
(10) Pub. No.: US 2025/0073401 A1
(43) Pub. Date: Mar. 6, 2025

(54) COUNTER STRUCTURE OF DRY POWDER AEROSOL DELIVERY SYSTEM

(71) Applicant: SHANGHAI XIUXIN CHENPON PHARMACEUTICAL CO., LTD., Shanghai (CN)

(72) Inventors: Zhonghua TAN, Shanghai (CN); Ran WANG, Shanghai (CN); Yan ZHANG, Shanghai (CN); Guojun LUO, Shanghai (CN); Dizheng DU, Shanghai (CN)

(73) Assignee: SHANGHAI XIUXIN CHENPON PHARMACEUTICAL CO., LTD., Shanghai (CN)

(21) Appl. No.: 18/723,908

(22) PCT Filed: Jun. 16, 2022
(86) PCT No.: PCT/CN2022/099105

§ 371 Date: Jun. 25, 2024

(30) Foreign Application Priority Data
Dec. 31, 2021 (CN) 202111682931.0

(51) Int. Cl. A61M 15/00

(52) U.S. Cl. CPC A61M 15/0071

(57) ABSTRACT

Disclosed is a counter structure of a dry powder aerosol delivery system, belonging to the field of inhalation devices; the counter structure includes a housing; a knob is arranged at one end of the housing; a counting disc, a transmission gear, a counting disc cover, a spring, a vibration gear and a rotating disc are arranged in the housing; the housing, the knob, the counting disc, the counting disc cover, the spring, the vibration gear and the rotating disc are arranged coaxially; the counting disc, the counting disc cover, the spring and the vibration gear are disposed around a straight rod of the rotating disc; an outer gear meshed with the transmission gear is arranged on the periphery of the straight rod.

### TECHNICAL FIELD

The invention belongs to the field of devices for delivering media into human bodies, and particularly relates to a counter structure of a manually operated multi-dose inhaler.

### BACKGROUND

Dry powder aerosols are widely used for treating diseases such as asthma and chronic obstructive pulmonary diseases and are provided to patients as drug-device combination products, a dry powder inhalation device of which plays an important role in effectively delivering drugs and guaranteeing the treatment compliance of patients.

Drugs of reservoir-type dry powder aerosols, as multi-dose dry powder aerosols, generally need to be stored for a period of time, so the counter is used as a structure for displaying remaining doses in a device.

The counter of existing reservoir-type multi-dose dry powder aerosol inhalation devices (abbreviated as reservoir-type inhalers) is generally arranged on a fixed structure, and the structure of such a counter is not applicable to a moving part.

In a case where the counter is arranged on a fixed part, all what needs to be addressed is the change of scales of the counter. However, in a case where the counter is arranged on a movable part, relative changes of two movable parts need to be addressed, or counting is inaccurate due to a large counting interval of the counter. For 100 times of inhalation, if each time of inhalation is counted by a reading, a patient can accurately know the remaining times of inhalation; if every ten times of inhalation (or ten inhalation times) are counted by a reading, the interval between readings is large, and the remaining times of inhalation have to be estimated by the patient.

How to arrange a counter on a moving part of reservoir-type multi-dose dry powder aerosol inhalation devices to prevent remaining inhalation times from being estimated by patients is an actual technical issue that needs to be urgently settled in design and fabrication of the reservoir-type multi-dose dry powder aerosol inhalation devices.

### SUMMARY

The technical issue to be settled by the invention is to provide a counter structure of a dry powder aerosol delivery system. The counter structure can count and display remaining doses while distributing doses by means of a knob.

The technical solution of the invention is as follows: a counter structure of a dry powder aerosol delivery system comprises a housing of a dry powder aerosol delivery system, wherein:

A knob is arranged at one end of the housing; a counting disc, a transmission gear, a counting disc cover, a spring, a vibration and a rotating disc are arranged in the housing; the housing, the knob, the counting disc, the counting disc cover, the spring, the vibration gear and the rotating disc are arranged coaxially; the counting disc, the counting disc cover, the spring and the vibration gear are disposed around a straight rod of the rotating disc; an outer gear meshed with the transmission gear is arranged on the periphery of the straight rod; a counter window is formed on a front surface of the knob to allow a patient to see readings and obtain used inhalation times or available remaining inhalation times; a counter function is realized on the knob of the counter structure, such that doses are be distributed by means of the knob, and the remaining doses are counted and displayed.

Specifically, a limiting groove is formed in an outer wall of the housing, unidirectional grooves are formed in an inner wall of the housing, one surface of each unidirectional groove is a straight surface, and the other surface of each unidirectional groove is an oblique surface.

Specifically, the counting disc and the transmission gear are arranged in the knob.

Further, a gear meshed with the transmission gear is arranged in the counting disc.

Further, a center shaft is arranged on the transmission gear, an upper end of the center shaft is limited on the counting disc cover by means of a hole, and a lower end of the center shaft is limited on the knob by means of a hole; and two gears are arranged in a middle of the center shaft of the transmission gear, an upper gear is meshed with the rotating disc, a lower gear is meshed with the counting disc, and the upper gear and the lower gear are arranged coaxially.

Specifically, the counting disc cover is clamped on an inner wall of the knob and rotates synchronously with the knob, a center hole of the counting disc cover is used for limiting the rotating disc, and uniformly-distributed groove structures are formed in an upper surface of the counting disc cover to allow the vibration gear to rotate synchronously with the counting disc cover.

Further, the spring is arranged between the counting disc cover and the vibration gear; and first bevel vibration teeth are arranged on a top surface of the vibration gear and matched with second bevel vibration teeth arranged on a bottom surface of the rotating disc, potential energy of the spring is stored by means of bevel surfaces of the first and second bevel vibration teeth and is released by means of straight surfaces of the first and second bevel vibration teeth to provide a vibration force.

Specifically, unidirectional pawls are arranged on a periphery of the rotating disc, one surface of each unidirectional pawl is a straight surface, and the other surface of each unidirectional pawl is an oblique surface, such that the rotating disc is only allowed to rotate along the oblique surfaces in one direction and is not allowed to rotate along the straight surfaces in an opposite direction; and the straight rod is arranged at a center of the rotating disc, and the straight rod downward extends to the transmission gear and is meshed with the transmission gear by means of the outer gear arranged on the straight rod.

According to the technical solution, the counter structure is operated and used as follows:

Process 1: a patient rotates the knob to drive the counting disc cover and the vibration gear to rotate, the rotating disc keeps static under the action of the unidirectional pawls, the vibration gear moves downwards under the action of bevel teeth, and the spring is compressed; when the knob is limited by a lower housing after rotating to an angle, vibration teeth turn from bevel teeth into straight teeth, the vibration gear returns to an original position under the action of the spring, and in the process, the counting disc rotates synchronously with the knob, so a reading is not changed;

Process 2: the patient rotates the knob to the initial position, the knob drives the counting disc cover and the vibration gear to rotate, the vibration gear drives the rotating disc to rotate by means of the straight teeth, and during the rotation process of the rotating disc, the transmission gear drives the counting disc to rotate by means of the gear at a bottom, and the rotation of the counting disc leads to a change of the reading displayed by the counter window.

Further, every time the patient completes one time of drug application, the counter structure counts backward once until the reading of the counter structure becomes zero, that is, a service life of the dry powder aerosol delivery system ends.

### BRIEF DESCRIPTION OF THE SEVERAL VIEWS OF THE DRAWINGS

FIG. 1a and FIG. 1b are schematic structural views of a lower housing according to the invention;

FIG. 2a and FIG. 2b are schematic structural views of a knob according to the invention;

FIG. 3a and FIG. 3b are schematic structural views of a counting disc according to the invention;

FIG. 4a and FIG. 4b are schematic structural views of a transmission gear according to the invention;

FIG. 5a and FIG. 5b are schematic structural views of a counting disc cover according to the invention;

FIG. 6 is a schematic structural view of a spring according to the invention;

FIG. 7a and FIG. 7b are schematic structural views of a vibration gear according to the invention;

FIG. 8a and FIG. 8b are schematic structural views of a rotating disc according to the invention;

FIG. 9 is an overall structural view of a counter structure after assembly;

FIG. 10 is an overall sectional structural view of the counter structure;

FIG. 11a and FIG. 11b are schematic diagrams of counting readings.

In the FIGS.: 1, lower housing; 1-1, limiting groove; 1-2, unidirectional groove; 2, knob; 2-1, counter window; 3, counting disc; 3-1, gear; 4, transmission gear; 4-1, upper gear; 4-2, lower gear; 5, counting disc cover; 5-1, center hole; 5-2, upper surface; 5-3, uniformly-distributed groove structure; 6, Spring; 7, vibration gear; 7-1, first bevel vibration tooth; 8, rotating disc; 8-1, second bevel vibration tooth; 8-2, unidirectional pawl; 8-3, straight rod; 8-4, outer gear.

### DETAILED DESCRIPTION

The invention is further described below in conjunction with accompanying drawings.

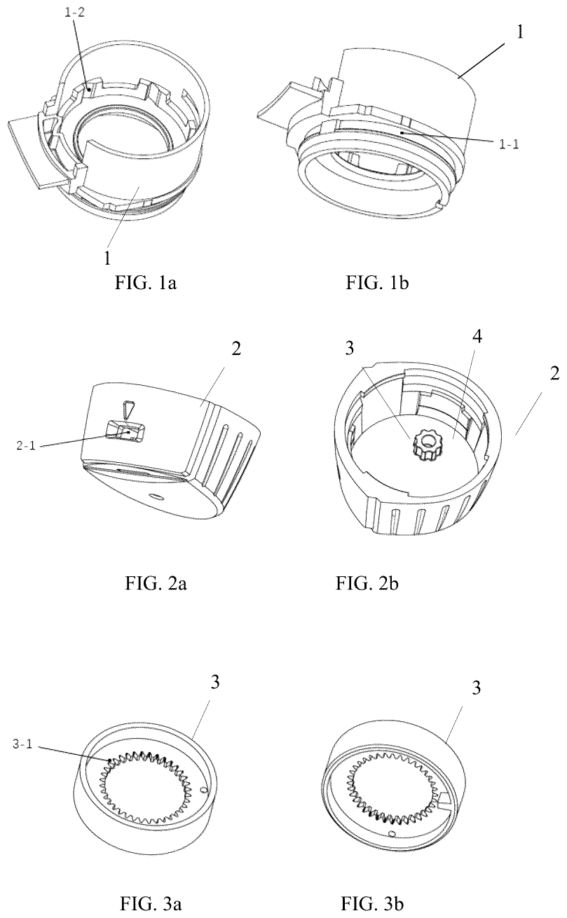

As shown in FIG. 1a to FIG. 10, a counter structure in the technical solution of the invention is one part of a reservoir-type inhaler and is composed of a lower housing 1, a knob 2, a counting disc 3, a transmission gear 4, a counting disc cover 5, a spring 6, a vibration gear 7 and a rotating disc 8.

The lower housing is defined merely according to the direction in the drawings, and a housing of the counter structure is not definitely a lower housing. More accurately, the lower housing is arranged at one end of the reservoir-type inhaler, and thus to be more precisely, should be referred to as a housing.

The housing, the knob, the counting disc, the counting disc cover, the spring, the vibration gear and the rotating disc are arranged coaxially.

The counting disc, the counting disc cover, the spring and the vibration gear are disposed around a straight rod of the rotating disc; and an outer gear meshed with the transmission gear is arranged on the periphery of the straight rod.

Referring to FIG. 1a and FIG. 1b, a limiting groove 1-1 is formed in an outer wall of the lower housing 1, such that the knob 2 can rotate repeatedly by a certain angle in the limiting groove; unidirectional grooves 1-2 are formed in an inner wall of the lower housing 1, such that unidirectional pawls 8-2 of the rotating disc 8 can rotate in the unidirectional grooves in the same direction; one surface of each unidirectional groove 1-2 is a straight surface and the other surface of each unidirectional groove 1-2 is an oblique surface (as shown in FIG. 1a), and the unidirectional pawls 8-2 can slide out along the oblique surfaces when rotating clockwise and will be blocked by the straight surfaces when rotating anticlockwise.

In FIG. 2a and FIG. 2b, a counter window 2-1 is formed on a front surface of the knob 2 to allow a patient to see readings; and the counting disc 3 is arranged in the knob and rotates together with the transmission gear 4.

Readings are printed on an outer surface of the counting disc 3 according to the total number of doses of a drug and gradually decrease from the total number of doses to 0, and the readings corresponding to the last ten inhalation times are printed in red for warning.

In FIG. 3a and FIG. 3b, a gear 3-1 is arranged in the counting disc 3 and meshed with the transmission gear 4.

A center shaft 4a is arranged on the transmission gear 4, an upper end of the center shaft is limited on the counting disc cover 5 by means of a hole, and a lower end of the center shaft is limited on the knob 2 by means of a hole.

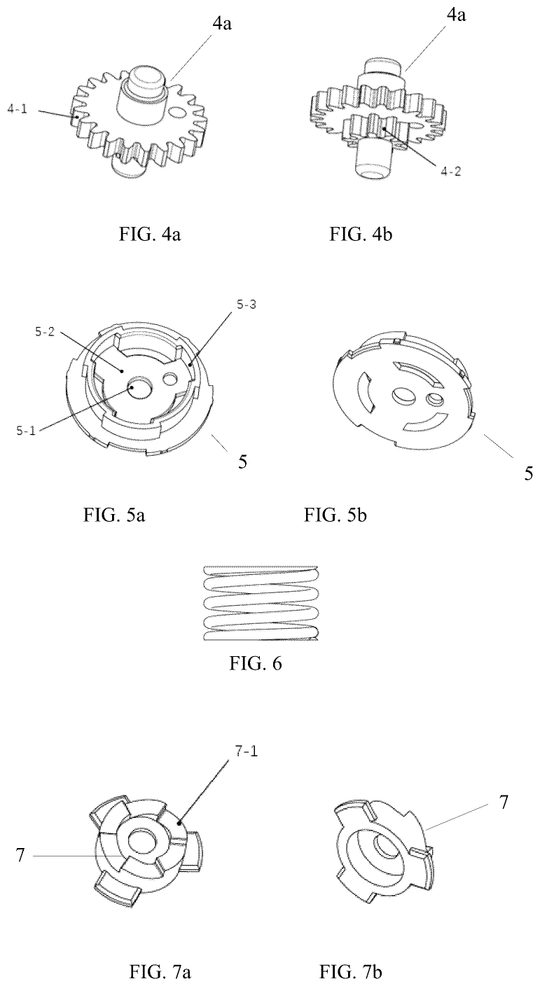

As shown in FIG. 4a and FIG. 4b, two gears are arranged in the middle of the center shaft 4a of the transmission gear 4, an upper gear 4-1 is meshed with the rotating disc, a lower gear 4-2 is meshed with the counting disc, and the upper gear and the lower gear are arranged coaxially.

In FIG. 5a and FIG. 5b, because the counting disc cover is clamped on the inner wall of the knob, the counting disc cover can rotate synchronously with the knob, a center hole 5-1 of the counting disc cover is used for limiting the rotating disc 8, and uniformly-distributed groove structures 5-3 are formed in an upper surface 5-2 of the counting disc cover and allow the vibration gear to be clamped therein, such that the vibration gear can rotate synchronously with the counting disc cover.

As show in FIG. 6, the spring 6 is arranged between the counting disc cover and the vibration gear and limited by vibration teeth. During operation, the spring can be compressed and released instantly to prove elastic potential energy.

In FIG. 7a and FIG. 7b, first bevel vibration teeth 7-1 are arranged on a top surface of the vibration gear 7 and matched with second bevel vibration teeth 8-1 arranged on a bottom surface of the rotating disc. The potential energy of the spring is stored by means of bevel surfaces of the first and second bevel vibration teeth and is released by means of straight surfaces of the first and second bevel vibration teeth to provide a vibration force.

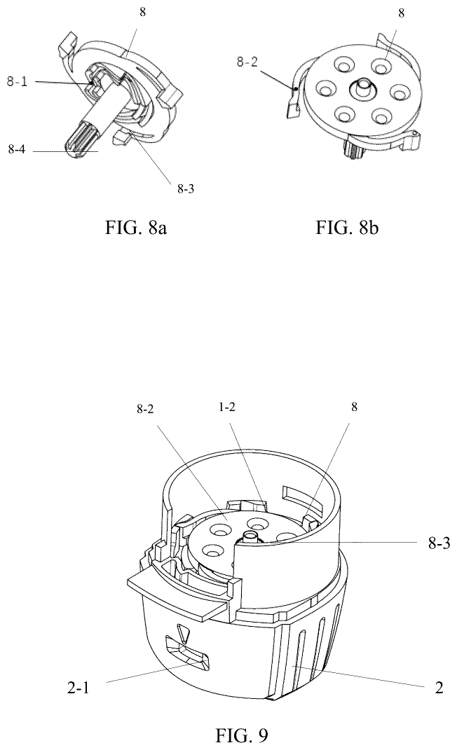

As shown in FIG. 8a and FIG. 8b, unidirectional pawls 8-2 are arranged on the periphery of the rotating disc 8, one surface of each unidirectional pawl 8-2 is a straight surface, and the other surface of each unidirectional pawl 8-2 is an oblique surface, such that the rotating disc is only allowed to rotate along the oblique surfaces in one direction and is not allowed to rotate along the straight surfaces in an opposite direction.

A straight rod 8-3 is arranged at the center of the rotating disc 8, and the straight rod downward extends to the transmission gear and is meshed with the transmission gear 4 by means of an outer gear 8-4 arranged on the straight rod.

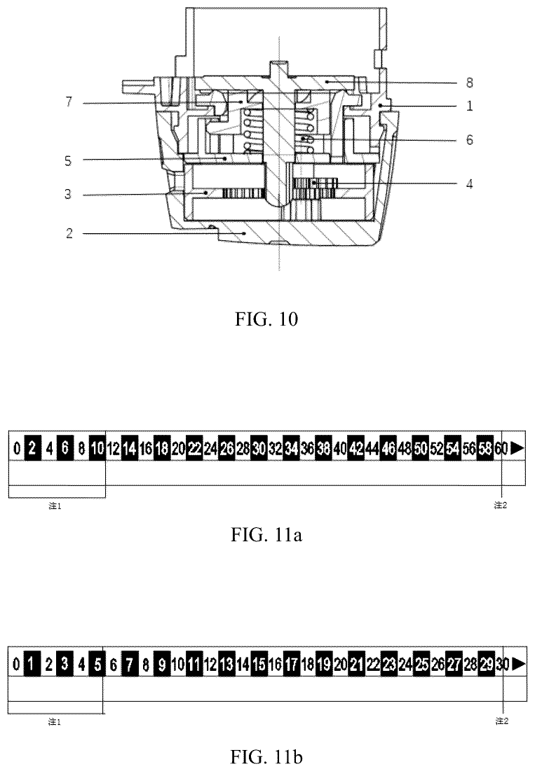

FIG. 9 and FIG. 10 illustrate the overall structure and sectional structure of the counter structure after assembly.

FIG. 11a and FIG. 11b are schematic diagrams of counting readings according to the technical solution, wherein FIG. 11a illustrates an example where the maximum number of times of inhalation is 60, and FIG. 11b illustrates an example where the maximum number of times of inhalation is 30.

In addition, as shown by annotation 1 in FIG. 11a and FIG. 11b, the inside of the box is printed in white on a red background, and the outside of the box is printed in red on a white background.

As shown by annotation 2 in FIG. 11a and FIG. 11b, after the counter structure is assembled, the initial position of the counter structure is aligned with a midline of the counter window.

By means of the structural design of the above components and the cooperation between these components, the technical solution can fulfill the following effects:

Process 1: a patient rotates the knob to drive the counting disc cover and the vibration gear to rotate, the rotating disc keeps static under the action of the unidirectional pawls, the vibration gear moves downwards under the action of the bevel teeth, and the spring is compressed; when the knob is limited by the lower housing after rotating to a certain angle, the vibration teeth turn from bevel teeth into straight teeth, the vibration gear returns to the original position under the action of the spring, and in this process, the counting disc rotates synchronously with the knob, so the reading is not changed;

Process 2: the patient rotates the knob to the initial position, the knob drives the counting disc cover and the vibration gear to rotate, the vibration gear drives the rotating disc to rotate by means of the straight teeth, and during the rotation process of the rotating disc, the transmission gear drives the counting disc to rotate by means of the gear at the bottom, and the rotation of the counting disc leads to a change of the readings;

Every time the patient completes one time of drug application, the counter structure counts backward once until the reading of the counter structure becomes zero, that is, the service life of a dry powder aerosol delivery system ends.

### EMBODIMENTS

### Embodiment 1

When the knob rotates by 30°, the speed is changed three times by means of the rotating disc and the counting disc at the first transmission stage, the second transmission stage is not set, the rotating disc rotates by 10°, and the counting readings are printed from 36 to 0, preferably from 30 to 0.

### Embodiment 2

When the knob rotates by 30°, the speed is changed three times by means of the rotating disc and the transmission gear at the first transmission stage and is changed twice by means of the transmission gear and the counting disc at second transmission stage, the rotating disc rotates by 5°, and the counting readings are printed from 72 to 0, preferably from 60 to 0.

### Embodiment 3

When the knob rotates by 60°, the speed is changed three times by means of the rotating disc and the transmission gear at the first transmission stage and is changed twice by means of the transmission gear and the counting disc at second transmission stage, the rotating disc rotates by 10°, and the counting readings are printed from 36 to 0, preferably from 30 to 0.

### Embodiment 4

When the knob rotates by 60°, the speed is changed three times by means of the rotating disc and the transmission gear at the first transmission stage and is changed four times by means of the transmission gear and the counting disc at second transmission stage, the rotating disc rotates by 5°, and the counting readings are printed from 72 to 0, preferably from 30 to 0.

### Embodiment 5

When the knob rotates by 90°, the speed is changed three times by means of the rotating disc and the transmission gear at the first transmission stage and is changed three times by means of the transmission gear and the counting disc at second transmission stage, the rotating disc rotates by 10°, and the counting readings are printed from 36 to 0, preferably from 30 to 0.

### Embodiment 6

When the knob rotates by 90°, the speed is changed three times by means of the rotating disc and the transmission gear at the first transmission stage and is changed six times by means of the transmission gear and the counting disc at second transmission stage, the rotating disc rotates by 5°, and the counting readings are printed from 72 to 0, preferably from 60 to 0.

### Embodiment 7

When the knob rotates by 90°, the speed is changed six times by means of the rotating disc and the transmission gear at the first transmission stage and is changed six times by means of the transmission gear and the counting disc at second transmission stage, the rotating disc rotates by 2.5°, and the counting readings are printed from 144 to 0, preferably from 120 to 0.

### Embodiment 8

When the knob rotates by 180°, the speed is changed three times by means of the rotating disc and the transmission gear at the first transmission stage and is changed six times by means of the transmission gear and the counting disc at second transmission stage, the rotating disc rotates by 10°, and the counting readings are printed from 36 to 0, preferably from 30 to 0.

### Embodiment 9

When the knob rotates by 180°, the speed is changed six times by means of the rotating disc and the transmission gear at the first transmission stage and is changed six times by means of the transmission gear and the counting disc at second transmission stage, the rotating disc rotates by 5°, and the counting readings are printed from 72 to 0, preferably from 60 to 0.

### Embodiment 10

The counting readings can record remaining doses and are printed one by one in a descending order, for example, 60, 59, 58, . . . , 0; or the counting readings are printed at intervals, for example, 60, 58, 56, . . . , 0; the counting reads can also record the number of times of use in an ascending order, for example, 0, 1. 2, . . . , 60.

To sum up, compared with the prior art, the technical solution of the invention has the following advantages:
1. According to the technical solution, a counter is arranged on a knob of a dry powder aerosol inhalation device, such that patients can know remaining doses in the device;
2. Two sets of meshed gears are used for drive, the rotation angle of the knob located outside and the rotation angle of the counting disc located inside can be adjusted as needed, thus guaranteeing the universality;
3. The counter is arranged on the moving knob, thus breaking through the limitation of the arrangement position of the counter.

The above embodiments are merely used for explaining and describing the invention, and are not intended to limit the technical solution of the invention. Those ordinarily skilled in the art should appreciate that all transformations and modifications made to the above embodiments based within the scope of the essential spirit of the invention should also fall within the protection scope of the claims of the invention.

### INDUSTRIAL APPLICATION

The technical solution of the invention provides a counting functional module or unit for a reservoir-type multi-dose drug delivery system, which allows patients to rotate a knob repeatedly to realize transmission by means of unidirectional processing (reciprocation of the knob and rotation of the counting disc in one direction) and speed changing (rotation of the knob by a certain angle, two stages of speed change with gears, and rotation of the counter by a different angle) to obtain reading information of used inhalation times or remaining inhalation times based on digital printing.

In the technical solution, the rotation angle of the knob can be set as needed, and the total number of times of inhalation can be set as needed through transmission speed-change.

In the invention, the readings of the counter can be printed in an ascending order or a descending order, one by one, or at intervals as needed.

In the invention, the counter is arranged on a moving part (the knob), the readings will not be displayed mistakenly during the movement process of the moving part, and only one reading is changed every time the knob is rotated once.

The counter structure provided by the invention is suitable for a reservoir-type dry powder inhaler, and is especially suitable for a reservoir-type dry powder inhaler device, the counter of which cannot be arranged on a static part, or a reservoir-type dry powder inhaler device, the counter function of which can only be set on a moving part due to the limitation of space and structure.

The counter structure provided by the invention can be widely used in the field of design and fabrication of reservoir-type dry powder inhalers.

What is claimed is:

1. A counter structure of a dry powder aerosol delivery system, comprising a housing of a dry powder aerosol delivery system, wherein:
a knob is arranged at an end of a housing;
a counting disc, a transmission gear, a counting disc cover, a spring, a vibration gear and a rotating disc are arranged in the housing;
the housing, the knob, the counting disc, the counting disc cover, the spring, the vibration gear and the rotating disc are arranged coaxially;
the counting disc, the counting disc cover, the spring and the vibration gear are disposed around a straight rod of the rotating disc; an outer gear meshed with the transmission gear is arranged on a periphery of the straight rod;
a counter window is formed on a front surface of the knob to allow a patient to see readings and obtain used inhalation times or available remaining inhalation times;
a counter function is realized on the knob of the counter structure, such that doses are be distributed by means of the knob, and the remaining doses are counted and displayed.

2. The counter structure of a dry powder aerosol delivery system according to claim 1, wherein a limiting groove is formed in an outer wall of the housing, unidirectional grooves are formed in an inner wall of the housing, one surface of each said unidirectional groove is a straight surface, and the other surface of each said unidirectional groove is an oblique surface.

3. The counter structure of a dry powder aerosol delivery system according to claim 1, wherein the counting disc and the transmission gear are arranged in the knob.

4. The counter structure of a dry powder aerosol delivery system according to claim 1, wherein a gear meshed with the transmission gear is arranged in the counting disc.

5. The counter structure of a dry powder aerosol delivery system according to claim 1, wherein a center shaft is arranged on the transmission gear, an upper end of the center shaft is limited on the counting disc cover by means of a hole, and a lower end of the center shaft is limited on the knob by means of a hole;
two gears are arranged in a middle of the center shaft of the transmission gear, an upper gear is meshed with the rotating disc, a lower gear is meshed with the counting disc, and the upper gear and the lower gear are arranged coaxially.

6. The counter structure of a dry powder aerosol delivery system according to claim 1, wherein the counting disc cover is clamped on an inner wall of the knob and rotates synchronously with the knob, a center hole of the counting disc cover is used for limiting the rotating disc, and uniformly-distributed groove structures are formed in an upper surface of the counting disc cover to allow the vibration gear to rotate synchronously with the counting disc cover.

7. The counter structure of a dry powder aerosol delivery system according to claim 1, wherein the spring is arranged between the counting disc cover and the vibration gear;
first bevel vibration teeth are arranged on a top surface of the vibration gear and matched with second bevel vibration teeth arranged on a bottom surface of the rotating disc, potential energy of the spring is stored by means of bevel surfaces of the first and second bevel vibration teeth and is released by means of straight surfaces of the first and second bevel vibration teeth to provide a vibration force.

8. The counter structure of a dry powder aerosol delivery system according to claim 1, wherein unidirectional pawls are arranged on a periphery of the rotating disc, one surface of each said unidirectional pawl is a straight surface, and the other surface of each said unidirectional pawl is an oblique surface, such that the rotating disc is only allowed to rotate along the oblique surfaces in one direction and is not allowed to rotate along the straight surfaces in an opposite direction;
the straight rod is arranged at a center of the rotating disc, and the straight rod downward extends to the transmission gear and is meshed with the transmission gear by means of the outer gear arranged on the straight rod.

9. The counter structure of a dry powder aerosol delivery system according to claim 1, the counter structure is operated and used as follows:
process 1: a patient rotates the knob to drive the counting disc cover and the vibration gear to rotate, the rotating disc keeps static under the action of the unidirectional pawls, the vibration gear moves downwards under the action of bevel teeth, and the spring is compressed; when the knob is limited by a lower housing after rotating to an angle, vibration teeth turn from bevel teeth into straight teeth, the vibration gear returns to an original position under the action of the spring, and in the process, the counting disc rotates synchronously with the knob, so a reading is not changed;
process 2: the patient rotates the knob to the initial position, the knob drives the counting disc cover and the vibration gear to rotate, the vibration gear drives the rotating disc to rotate by means of the straight teeth, and during the rotation process of the rotating disc, the transmission gear drives the counting disc to rotate by means of the gear at a bottom, and the rotation of the counting disc leads to a change of the reading displayed by the counter window.

10. The counter structure of a dry powder aerosol delivery system according to claim 9, wherein every time the patient completes one time of drug application, the counter structure counts backward once until the reading of the counter structure becomes zero, that is, a service life of the dry powder aerosol delivery system ends.
